(19) United States
(12) Patent Application Publication
(10) Pub. No.: US 2008/0045881 A1
(43) Pub. Date: Feb. 21, 2008

(54) DEVICES AND METHODS FOR REMOVING A MATTER FROM A BODY CAVITY OF A PATIENT

(75) Inventors: George P. Teitelbaum, Santa Monica, CA (US); Donald W. Larsen, La Canada, CA (US)

Correspondence Address: HOGAN & HARTSON L.L.P., 1999 AVENUE OF THE STARS, SUITE 1400, LOS ANGELES, CA, 90067, US

(73) Assignee: University of Southern California, Los Angeles, CA (US)

(21) Appl. No.: 11/829,879

(22) Filed: Jul. 27, 2007

Related U.S. Application Data
(63) Continuation-in-part of application No. 11/087,780, filed on Mar. 23, 2005.
(63) Continuation-in-part of application No. PCT/US05/09658, filed on Mar. 24, 2005.
(60) Provisional application No. 60/556,993, filed on Mar. 26, 2004.
(60) Provisional application No. 60/611,684, filed on Sep. 20, 2004.
(60) Provisional application No. 60/820,671, filed on Jul. 28, 2006.

(51) Int. Cl. A61M 25/00

(52) U.S. Cl. 604/21; 604/509

(57) ABSTRACT

Disclosed are systems and methods for removing a matter from a body cavity of a patient. Exemplary systems of the present invention generally comprise a delivery catheter device, a central matter retrieval device and a device retriever. The central matter retrieval device has an elongated body and shape memory foam attached thereon. In use, the central matter retrieval device is housed within a parking segment of the delivery catheter and delivered to a desired body cavity in a patient. The retriever device is separately deployed to securely remove the matter and the central matter retrieval device from the patient.

This application is a continuation-in-part of U.S. patent application Ser. No. 11/087,780, filed Mar. 23, 2005, and PCT US/05/009658, filed Mar. 24, 2005, both of which claim the benefit of U.S. Provisional Application No. 60/556,993, filed on Mar. 26, 2004 and 60/611,684, filed on Sep. 20, 2004. Furthermore, this application also claims priority to the U.S. Provisional Patent Application No. 60/820,671, filed on Jul. 28, 2006. The content of these applications are incorporated herein by reference in their entirety.

### FIELD OF THE INVENTION

This invention relates to devices and methods for removing a matter from a body cavity of a patient and delivery of a therapeutic agent. In particular, the invention is directed to devices, including, but not limited to, endovascular devices, comprising a radially expandable polymer for engaging and removing the matter.

### BACKGROUND OF THE INVENTION

A number of vascular disorders, such as stroke, pulmonary embolism, peripheral thrombosis, atherosclerosis, and the like, are characterized by formation of occlusions that prevent normal blood flow in blood vessels. For example, an ischemic stroke is a neurological dysfunction caused by a blockage of one of the major blood arteries of the brain. The blockage can be the result of the formation of blood clot at the site of blockage (thrombosis), obliteration of the lumen of a blood vessel due to atherosclerosis, or the migration of an occluding blood clot (formed in the heart, carotid artery, or elsewhere) downstream to the site of blockage (embolization).

Clot-busting (thrombolytic) drugs have been employed to break up clots blocking a particular blood vessel. But the success rate of this approach is still quite low. For example, at present, the only FDA-approved thrombolytic drug for acute (less than three hours old) ischemic stroke is tissue plasminogen activator (tPA). With this form of therapy, only 30% of patients are expected to realize a good or excellent clinical outcome several months following infusion, and patients who demonstrate signs of intracranial hemorrhage at the time of presentation (on a head CT study) are not candidates for tPA therapy. Also, intravenous tPA therapy is associated with an almost 6% fatal intracranial hemorrhage rate. Due to these shortcomings, there has been increasing interest in the development of a mechanical means of clot retrieval or dissolution. Concentric Medical, Inc. (Mountain View, Calif.) has created an intraluminal clot retrieval system comprised of a nitinol (Nickel-Titanium alloy) shape-memory corkscrew-like coil that is advanced into an occluding clot (U.S. Pat. Nos. 5,895,398; 6,638,245; 6,530,935; and 6,692,509). The coil and its attached wire are then withdrawn from the affected cerebral vessel, retrieving the thrombus material into a balloon-tipped guiding catheter positioned in the internal carotid artery. This device has been shown, in a prospective nonrandomized human clinical study (MERCI Trial), to achieve a 53.5% revascularization rate with a serious device and/or procedure-related adverse event rate of 7%. There was a 31% death rate in the recanalized patients vs. a 57% death rate in the nonrecanalized patients. There was an 8% symptomatic intracerebral hemorrhage rate (lower than the 10% intracranial hemorrhage rate experienced during the intra-arterial thrombolysis PROACT II trial).

Although the results are promising, the Concentric Medical's clot retrieval device suffered from an approximately 6% wire breakage rate. Thus, there still exists an unfulfilled need for safer, more reliable, and effective mechanical clot retrieval devices. More generally, there is a need for reliable, safe, and effective devices and methods of retrieving a matter from a body cavity of a human or an animal patient.

### SUMMARY OF THE INVENTION

Accordingly, an object of the present invention is to provide devices and methods for engaging and removing a matter from a body cavity of a patient, including endovascular devices and methods for removing a matter from a lumen of a blood vessel. Also, it is an object of the invention to provide devices and methods for delivery of therapeutic agents.

These and other objects are achieved in the device of the present invention. The device comprises an elongated carrier having a distal portion adapted for positioning inside a body cavity and a proximate portion. A radially expandable polymer is circumferentially attached to the distal portion of the carrier and adapted to enter a matter located inside the body cavity while in a compressed configuration. The expandable polymer is capable of transitioning to an expanded configuration while inside the matter to penetrate and engage it from within.

The body cavity may be a naturally existing or surgically made conduit or cavity. Examples of such conduits and cavities include, but are not limited to, blood vessels; parts of the alimentary tract including esophagus, stomach, small and large bowel, anus and rectum; parts of the genitourinary system including renal pelvis, ureter, urethra, spermatic cord, fallopian tubes; the ventricles and cisterns of the brain; the urinary bladder; cysts, vagina; uterine cavity; pseudocysts; abcesses; and fistuae.

The transition of the polymer between the compressed configuration and the expanded configuration may be triggered by a physiological or an external stimulus. Examples of the physiological stimulus include, but are not limited to, body temperature, blood pH, an ion concentration in blood, and blood composition. Examples of the external stimulus include, but are not limited to, changes in local chemical environment, changes in external temperature, light, magnetic field, ultrasound, radiation, and electrical field. For example, a biocompatible solution may be introduced into the blood vessel which causes changes in the local chemical environment and results in the expansion of the polymer.

In one embodiment, the polymer is a hydrogel and the transition of the hydrogel into the expanded configuration is triggered by a hydration of the hydrogel or by application of a triggering fluid to the hydrogel. In another embodiment, the polymer is a shape memory foam. For example, the shape memory foam may have an original expanded configuration that is compressed at a temperature above a glass transition temperature, Tg, to form the compressed configuration. The foam retains its compressed configuration at a temperature below Tg but returns substantially to its original expanded configuration when it is exposed to a temperature above the Tg.

The polymer in its expanded configuration may have any shape and form as long as it allows to penetrate and engage the matter to be removed from within. For example, it may be in a form of a coil, a twisted ribbon, a screw-like structure, a disk, a sphere, a parachute-like structure, a formation comprising a plurality of ridges and troughs, and a formation comprising a plurality of outwardly extending spears. In one embodiment, the expanded configuration of the polymer has a twisted ribbon shape and the polymer is capable of storing torque energy and releasing it on demand.

In another aspect, the present invention provides another device for retrieving a matter from a body cavity of a patient. The device comprises an elongated carrier having a distal portion adapted for positioning inside the body cavity and at least two isolated formations of radially expandable polymer attached to the distal portion of the carrier. Each formation encloses the entire circumference of the carrier. The formations are adapted to move through or around the matter while having a compressed configuration and capable to transition to an expanded configuration to trap the matter therebetween. In one embodiment, the polymer is a hydrogel or a foam. The device may comprise a plurality of progressively decreasing in size formations of the radially expandable polymer. For example, the formations may be disks, spheres, outwardly extending spears, or configurations comprising a plurality of ridges and troughs.

In still another aspect, the present invention provides another device for retrieving a matter from a body cavity of a patient. The device comprises an elongated carrier having a distal portion adapted for positioning inside the body cavity and a radially expandable polymer circumferentially attached to the distal portion of the carrier. The polymer is adapted to move through or around the matter while having a compressed configuration and capable to transition to an expanded configuration to engage the matter for retrieval from the body cavity. In this embodiment of the invention, the transition of the polymer is triggered by a physiological stimulus.

In yet another aspect, the present invention provides a device with a retrieval element. The retrieval element is adapted for positioning inside a body cavity of a patient. The retrieval element has a proximal end and a distal end comprising an expandable sleeve. The retrieval element has a channel that extends through an entire length of the retrieval element and the expandable sleeve. The retrieval element further includes an inflatable balloon positioned concentrically inside the channel, the balloon, when inflated, is capable of radially expanding the expandable sleeve. The device further includes an elongated carrier slidably positioned within the channel of the retrieval element, wherein the elongated carrier has a distal portion adapted to move through the expandable sleeve of the retrieval element into the body cavity. The device also has a radially expandable polymer circumferentially attached to the distal portion of the carrier and adapted to move through or around the matter while having a compressed configuration and capable to transition to an expanded configuration to engage the matter. The expandable polymer in its expanded configuration is capable of being at least partially retrieved into the expandable sleeve.

The invention also provides a number of methods of retrieving a matter from a body cavity of a patient. In one embodiment, the method comprises (a) providing a device having a carrier with a distal portion and a radially expandable polymer circumferentially attached to the distal portion of the carrier, wherein the expandable polymer has an initial compressed configuration; (b) advancing the distal portion of the carrier of the device into the body cavity; (c) positioning the expandable polymer inside the matter; (d) allowing a sufficient time for the expandable polymer to transition from the initial compressed configuration to an expanded configuration whereby penetrating and engaging the matter from within; and (e) retrieving the device from the body cavity thereby removing the matter.

In another embodiment, the method comprises (a) providing a device having a carrier with a distal portion and at least two isolated formations of radially expandable polymer attached to the distal portion of the carrier, wherein each formation encloses the entire circumference of the carrier and the expandable polymer has an initial compressed configuration; (b) advancing the distal portion of the carrier into the body cavity; (c) passing at least one formation through or around the matter; (d) allowing sufficient time for the expandable polymer to transition from the initial compressed configuration to an expanded configuration whereby trapping the matter between the formations; and (d) retrieving the device thereby removing the matter.

In still another embodiment, the method of retrieving a matter from a body cavity of a patient comprises: (a) providing a device having a radially expandable polymer circumferentially attached to the distal portion of the carrier, wherein the expandable polymer has an initial compressed configuration; (b) positioning the distal portion of the carrier inside the body cavity and through or around the matter; (c) allowing sufficient time for a physiological stimulus to act on the expandable polymer to cause its transition from the initial compressed configuration to an expanded configuration whereby engaging the matter in a way that allows its removal; and (d) retrieving the device thereby removing the matter.

In yet another aspect, the invention provides a method of localized delivery of a therapeutic agent. The method comprises (a) providing a removable device having a carrier with a distal portion and a radially expandable polymer circumferentially attached to the distal portion of the carrier, wherein the expandable polymer has an initial compressed configuration; (b) advancing the distal portion of the carrier to a site in a body; and (c) allowing sufficient time for the expandable polymer to transition from the initial compressed configuration to an expanded configuration delivering the therapeutic agent. This method may be used to deliver a therapeutic drug anywhere in a body, including lumens, cavities, and solid tissue.

Finally, the invention provides a method of retrieving a matter from a body cavity of a patient utilizing a device with an expandable sleeve. The device comprises: (i) a retrieval element adapted for positioning inside the blood vessel, wherein the retrieval element has a proximal and a distal end, wherein the distal end comprises an expandable sleeve and wherein the retrieval element has a channel that extends through an entire length of the retrieval element and the balloon-expandable sleeve, the retrieval element further comprising an inflatable balloon positioned concentrically inside the channel, wherein the balloon, when inflated, is capable of radially expanding the expandable sleeve; (ii) an elongated carrier slidably positioned within the channel of the retrieval element, the elongated carrier having a distal portion; and (iii) a radially expandable polymer circumferentially attached to the distal portion of the carrier, wherein the expandable polymer has an initial compressed configuration.

The method comprises: (a) providing the device with the retrieval element; (b) positioning the retrieval element inside the body cavity; (c) advancing the distal portion of the carrier through the channel of the retrieval element and the expandable sleeve into the body cavity; (d) moving the distal portion of the carrier through or around the matter; (e) allowing sufficient time for the expandable polymer to transition from the initial compressed configuration to an expanded configuration whereby trapping the matter; (f) inflating the balloon whereby expanding the sleeve; and (g) retrieving, at least partially, the carrier with the expandable polymer in its expanded configuration into the expanded sleeve.

The above-described devices and methods of retrieval of a matter and delivery of a therapeutic agent provide a number of unexpected advantages over the existing devices and methods. The advantages include, but are not limited to, simple and economical yet reliable operation of the devices, which improves positive outcome of matter removal procedures. The use of a retrieval element according to one of the embodiments of the present invention further ensures safe retrieval of the matter from a body cavity.

Advantageously, the devices of the present invention accommodate attachment of optional steerable flexible tips that simplify navigation of the devices through body cavities such as the vasculature even to sites that are most remote from the entry point of the device. Also, expandable polymers (and foams in particular) used in the present invention allow more effective capturing of matter due to their better surface properties as compared to conventionally used metallic capture devices.

The invention is defined in its fullest scope in the appended claims.

### DESCRIPTION OF THE FIGURES

The above-mentioned and other features of this invention and the manner of obtaining them will become more apparent, and will be best understood by reference to the following description, taken in conjunction with the accompanying drawings, in which:

FIGS. 1A-1C schematically show several embodiments of the device of the present invention;

FIGS. 2A-2E schematically illustrate how the device shown in FIGS. 1A-1C may be used for removing a matter from a body cavity such as a lumen of a blood vessel;

FIGS. 3A-3H schematically show devices in accordance with other embodiments of the present invention; FIG. 3I shows forming a foam-like material from an expandable polymer in accordance with one embodiment of the present invention;

FIGS. 4A-4B schematically show devices in accordance with other embodiments of the present invention; FIGS. 4C-4E schematically illustrate how such devices may be used for removing a matter from a lumen of a blood vessel;

FIGS. 5A-5F show flexibility imparting features added to the expandable polymer in accordance with one embodiment of the present invention;

FIGS. 6A-6B depict an optional retrieval element with a self-deploying sleeve that may be used with devices of the present invention; FIGS. 6C-6E schematically illustrate how such device with the optional retrieval element may be used for removing a matter from a lumen of a blood vessel; FIGS. 6F-6G depict optional retrieval elements in accordance with other embodiments of the present invention;

FIGS. 7A-7F show an optional balloon-expandable retrieval element and its use for removing a matter from a blood vessel in accordance with an embodiment of the present invention.

FIGS. 8A-8B show devices of the present invention having a wire coil running through the expandable polymer in accordance with another embodiment of the present invention;

FIG. 9 shows delivery of a therapeutic agent into a solid tissue in accordance with one embodiment of the present invention.

### DETAILED DESCRIPTION OF THE INVENTION

Referring to FIGS. 1 and 2, in one aspect, the present invention is directed to a device 10 for removing a matter from a body cavity of a patient. The patient may be a human or an animal. The device 10 comprises an elongated carrier 12 having a distal portion 14 adapted to move through or within a body cavity of a patient, such as a lumen 42 of a blood vessel 40 and a proximate portion 16. A radially expandable polymer 20 is circumferentially attached to the distal portion 14 of the carrier 12 and adapted to enter a matter 50 blocking the lumen 42 while in a compressed configuration A shown in FIGS. 1A, 2A, and 2B.

It is to be understood that although FIG. 2 shows the device of the present invention being used to remove a matter from a blood vessel, the devices and methods of the present invention may be used in any conduit or cavity inside a patient's body that is naturally existing or surgically made. Examples of such conduits and cavities include, but are not limited to, parts of the alimentary tract including esophagus, stomach, small and large bowel, anus and rectum; parts of the genitourinary system including renal pelvis, ureter, urethra, spermatic cord, fallopian tubes; the ventricles and cisterns of the brain; the urinary bladder; cysts, vagina; uterine cavity; pseudocysts; abcesses; and fistuae. It is also to be understood that the form of the device depicted in FIGS. 1 and 2 has been chosen only for the purpose of describing a particular embodiment and function of the invention.

The device of the present invention may be used to remove any type of matter, including, but not limited to clots, emboli, calculi, pieces of atherosclerotic plaque and debris, loose pieces of tissue and neoplasia, inspissated thick secretions or fluids, and foreign bodies. The expandable polymer of the device engages the matter from within and drags it from its location into a larger retrieval/guiding catheter located within a body cavity. For example, in one embodiment of the present invention, the device is used to engage a clot in a blood vessel and drag it into a larger retrieval/guiding catheter located in the cervical internal carotid or vertebral artery.

The expandable polymer 20 is capable to transition to an expanded configuration B, which is shown, for example, in FIGS. 1B, 2C-2E, and 3A-3G, while inside the matter 50 to penetrate and engage it from within. The polymer may be attached to the carrier using any method of attachment that provides a reliable immobilization of the polymer on the carrier. Such methods are well-known and include, but are not limited to the use of a biocompatible epoxy adhesive, welding of a metal wire element running through the expandable polymer to the carrier, and/or trapping a collection of expandable polymer mechanically between two widened zones on the carrier. In one embodiment, the expandable polymer exhibits an adhesive property to the matter.

In one embodiment, the polymer is a shape memory polymer selected from a group consisting of polyurethane, polyethylene, polyethylene terephthalate, polyisoprene, styrene-butadiene copolymers, copolyester, ethylene-vinylacetate and other ethylene copolymers, acrylates including, but not limited polyacrylamide gel and polyacrylic acid, norbornane, polynorbornene, and polystyrenes. Using a shape memory polymer in the device of the present invention allows the device to be passed into the body cavity and navigated into the vicinity of a matter to be removed in a compressed configuration, which decreases a possibility of damaging the walls of the body cavity. For example, the device may be easily passed through a lumen of an intracranial microcatheter and subsequently to be navigated through or into the vicinity of a matter blocking a blood vessel without damaging the walls of the blood vessel.

The polymer may contain a predetermined amount of a therapeutic agent. In one embodiment the optional therapeutic agent is released when the polymer 20 transitions from the compressed configuration A to the expanded configuration B. For the purposes of the present invention, the phrase “therapeutic agent is released when the polymer 20 transitions” refers to a release of the therapeutic agent during or after the transition between the compressed configuration A and the expanded configuration B. One of the advantages of using the device of the present invention having a radially expandable polymer for drug delivery is a localized targeting of pathology and the avoidance of systemic delivery and undesirable systemic effects of a drug or vector.

The therapeutic agent is not limited to a particular chemical or biological group. Suitable therapeutic agents are well-known to physicians and are based on a patient's state of a disease. Some appropriate therapeutic agents include, but are not limited to an anti-thrombogenic, thrombolytic, anti-proliferative, anti-spasmodic, anticoagulant, anti-platelet adhesion drugs, endothelial cells, and gene vectors. In one embodiment, the thrombolytic drug is selected from a group consisting of tissue plasminogen activator (t-PA), streptokinase, a calcium ion influx inhibitor, urokinase, and their analogs.

The transition of the polymer between the compressed configuration A and the expanded configuration B may be triggered by a physiological stimulus, by an external stimulus, by a mechanical device or force, or by their combinations. Examples of the physiological stimulus include, but are not limited to, body temperature, blood pH, an ion concentration in blood, and overall blood composition. Examples of the external stimulus include, but are not limited to, solutions, introduction of which into the blood vessel causes changes in local chemical environment, changes in external temperature, light, magnetic field, ultrasound, radiation, and electrical field.

Examples of mechanical devices and forces include, but are not limited to, various types of sheathes, casings, covers, and other types of restrainers that are capable of retaining the expandable polymer in the compressed configuration. Removal of such restrainers leads to transition of the polymer into the expanded configuration. Referring to FIG. 1, in one embodiment, the device 10 of the present invention further comprises a delivery device 23 adapted for positioning inside the cavity and having an internal lumen 25, wherein the distal portion 14 of the carrier is slidably positioned within the lumen 25, wherein the polymer remains in the compressed configuration inside the delivery device and the polymer transitions into the expanded configuration when it exits the delivery device or delivery device is removed.

In one embodiment, the polymer is a hydrogel. A hydrogel is a three-dimensional network of hydrophilic polymer chains and water that fills the space between polymer chains. Typically, hydrogels are two- or multi-component systems, in which polymer chains are cross-linked through either chemical or physical bonding. In physical gels (pseudogels), the chains are connected by electrostatic forces, hydrogen bonds, hydrophobic interactions or chain entanglements. In chemical hydrogels, chains are linked by covalent bonds. Because of the hydrophilic nature of polymer chains, hydrogels absorb water and swell in the presence of abundant water. Typically, water constitutes at least 10% of the total weight (or volume) of a hydrogel.

Any hydrogels may be used for the purposes of the present invention as long as they are capable of transitioning from a compressed into an expanded configuration in a controllable fashion. Examples of hydrogels include, but are not limited to polyethylene oxide, polyvinyl alcohol, polyvinylpyrrolidone, polyhydroxyethyl methacrylate, polyetherpolycarbonatecollagen and polysaccharides.

The transition of the hydrogel of the present invention into the expanded configuration may be triggered by a number of internal and external stimuli, including, but not limited to, changes in hydration, pH, solute concentration (e.g., glucose concentration), ionic environment (including calcium, magnesium, potassium, and sodium), local light levels, temperature, electric field, magnetic field, radiation, and ultrasound. For example, in one embodiment, a hydrogel that swells at a predetermined time as a result of the absorption of blood from the blood vessel is used.

In another embodiment, a biocompatible triggering fluid is applied to a hydrogel to initiate the transition from the compressed to the expanded configuration. Triggering solutions are well-known in the art and may include fluids having a predetermined pH or composition that cause the hydrogel to swell and to transition into the expanded configuration. For example, lactated ringers solution, glucose, or saline may be used.

In another embodiment, the polymer is a shape memory foam. Shape memory polymer foams are materials that can be formed into a desired shape (“expanded configuration”) and then can be constrained into a deformed configuration (“compressed configuration”) at a temperature higher than the glass transition temperature point (Tg) of the polymer and then kept compressed at a temperature lower than the Tg. The original configuration of the foam can be at least partially recovered when the foam is again heated to and maintained at a temperature higher than the Tg.

Any shape memory foam may be used for the purposes of the present invention as long as it is capable of transitioning from a compressed into an expanded configuration in a controllable fashion. Examples of such foams include, but are not limited to polyurethane, a cross-linked ethylene-vinyl acetate, and polyethylene copolymers. Formulations and properties of shape memory foams are well-known to those skilled in the art and are described, for example, in the following references, each of which is incorporated herein by the reference: U.S. Pat. Nos. 5,049,591; 6,702,976; 5,032,622; 5,145,935; 5,188,792; 5,242,634; 5,418,261; 6,102,933; 6,156,842; 6,583,194; U.S. patent application Publ. No. US 2002/0101008 A1; Metcalfe et al., “Cold hibernated elastic memory foams for endovascular interventions;” Watt et al., “Thermomechanical properties of a shape memory polymer foam.” In one embodiment, a shape memory foam is a polyurethane foam. Such foams can be formulated to provide a desired Tg and cell size. In one embodiment, the foam's cell size is chosen to maximize its adhesiveness to the matter.

Although a variety of glass transition temperatures may be chosen, in one embodiment, Tg is below a body temperature (i.e., <37-38° C.) and the foam spontaneously transitions into the expanded configuration after being exposed to the body temperature for a predetermined time. In another embodiment, Tg is above a body temperature (i.e., >37-40° C.). In this embodiment, the device further comprises a source of heat 22. Those skilled in the art would recognize that a wide range of heat sources, including, but not limited to, electrical resistance, inductive, optical, and convective heating elements, may be used.

In one embodiment, the sources of heat is an electrical resistance element comprising a metal or a semi-conductive plastic coil 24 that is circumferentially attached to the distal portion 16 of the carrier 12 and electrically connected to a controller 26, which is located outside of the patient, through an insulated pathway. The controller 26 delivers direct electrical current at the appropriate voltage to the resistive heater to heat the foam layer to its Tg, thus enabling the foam to expand fully to its expanded configuration.

The controller is capable of adjusting a voltage applied to the coil 24 to maintain a predetermined temperature. The controller works by measuring the resistance within the circuit. This provides an indirect, but reliable measurement of the resistive heater's temperature, since as the heater's temperature rises, so does the circuit's resistance, in a predictable manner. Thus, as the circuit's resistance rises above an undesirable level, the controller shuts off current flowing to the heater. The controller will continue to assess the circuit's resistance by short bursts of current until the resistance falls to just below the critical level, at which point, direct current will again be delivered to the resistive heater at an appropriate voltage. The current flow continues until the critical resistance level is again exceeded, again terminating continuous current flow. This continuous feedback mechanism utilized by the heater controller maintains the heater's wire coil within a narrow temperature range around the foam's Tg.

Optionally, the heater controller may also include a timer that allows activation of the coil for an appropriate length of time, which is sufficient to ensure full expansion of the compacted foam segment. The heater's wire coil may be made of any metal or semi-conductive plastic. In one embodiment, tungsten is used.

The expandable polymer may be a material other than foam or hydrogel as long as it can be forced into a compressed configuration and is capable of transitioning into an expanded configuration. As shown in FIG. 3I, in one embodiment, cells, holes, and/or cavities 7 are machined by using a laser beam 9, a mechanical tool, or other means in a solid polymer 5 to impart foam-like texture and shape memory properties. Expandable polymers of the present invention may have a reticular pattern to increase their surface area for contact with the matter.

In one embodiment, the distal portion 14 of the elongated carrier 12 further comprises a steerable tip 28 (FIGS. 1A and 1C). The steerable tip may be a shapeable platinum or stainless steel wire. The optional steerable flexible tip of the present invention advantageously simplifies navigation of the device through the body cavity, such as vasculature, even to sites that are most remote from the entry point of the device.

The device 10 of the present invention may also serve to deliver catheters and other devices mounted on catheters in much the same way as an exchange guidewire. Examples of such devices may include angioplasty balloons, stents, or microcatheters. This may be a particularly useful feature if during attempted removal of an obstructing clot during treatment of acute stroke, a narrowing or stenosis in a blood vessel is discovered. When a stenosis is discovered while retrieving clot and retracting conventional Concentric Retreiver device having a “cork screw” configuration, the coil straightens out and the grip on the clot is lost. In addition, in some situations, the coil of the Concentric device could break off and or injure the blood vessel as attempts to drag it across a stenosis are made.

To ensure that matter being removed from the body cavity (e.g., a clot being removed from a blood vessel) is not lost when the device 10 is retrieved through a narrowed area, an angioplasty balloon, stent, or another similar device may be advanced over the proximal end of the carrier 12 and delivered to the site of the stenosis (downstream of the expandable polymer). The device may then be used to expand the narrowing and to enable removal of the clot retrieval device along with the matter. In one embodiment, the procedure described above may be used to perform an angioplasty to improve the luminal diameter of a narrowed blood vessel. The device 10 of the present invention may also serve as a protective filter in a blood vessel, distal to a site of angioplasty and/or stent placement, especially at intracranial sites, and have sufficient length to serve the function of an exchange wire while delivering angioplasty balloon catheters and stents to the treatment site.

The elongated carrier 12 of the present invention may be a guidewire or a catheter. For example, in one embodiment, a steerable guidewire with a preferred diameter range of 0.008″-0.018″, but possibly up to 0.038″ is used. The guidewire may be constructed of one or more fiber optic fibers, capable of transmitting light to the distal end of the device. In one embodiment, the light consists of laser light of one or different wavelengths and is capable of effecting a change in the expandable polymer configuration in one or more locations.

The polymer in its expanded configuration may have any shape and form as long as it allows to penetrate and engage the matter to be removed from within. For example, referring to FIGS. 3A-3G, it may be in a form of a coil or a screw-like structure (FIG. 3A), a twisted ribbon (FIG. 3F), a formation of one or more disks 32 (FIG. 3B), a parachute-like structure (FIG. 3E), a formation comprising a plurality of ridges 34 and troughs 36 (FIG. 3C), a formation of one or more spheres or globes 38 (FIG. 3D), and a formation comprising a plurality of outwardly extending spears 39 (FIG. 3G).

In several embodiments shown in FIGS. 3B, 3D, and 3G, the device comprises an elongated carrier 12 having a distal portion 14 adapted to move through the lumen and at least two isolated formations (e.g., 32 or 38) of radially expandable polymer attached to the distal portion of the carrier. Each formation encloses the entire circumference of the carrier. The formations are adapted to move through or around the matter while having a compressed configuration and capable to transition to an expanded configuration to trap the matter therebetween. In one embodiment, the polymer is a hydrogel or a foam.

The device may comprise a plurality of progressively decreasing in size formations of the radially expandable polymer. Such configuration advantageously permits the retrieval of clots, emboli, or foreign bodies from both larger and distally smaller vessels with the same device. The progressively decreasing in size formations may be disks (FIG. 3B), spheres (FIG. 3D), outwardly extending spears (FIG. 3G), or configurations comprising a plurality of ridges and troughs.

In one embodiment shown in FIG. 3E, the expanded configuration has a parachute-like structure surrounding and attached to the carrier 12. The parachute-like structure comprises a basket portion 44 for collecting the matter and at least two supporting struts 46, preferably, 2-6 supporting struts. The basket portion 44 has a hollow interior 54 and an opening 48 facing the proximate portion 16 of the carrier 12. The closed bottom 56 of the basket portion 44 is adjacent to the distal portion 14 of the carrier 12. The distal portion may optionally comprise a steerable shapeable tip 28. Also, optionally, the device may have an external source of heat with an electrical resistance element comprising a metal or a semi-conductive plastic coil 24.

Optionally, the struts may be reinforced by embedded wire loops or an embedded polymer fiber network 52 that would extend through the struts and into the distal cone portion of the parachute. In one embodiment, the wire loops are made of a shape memory material such as nitinol. In another embodiment, the polymer fiber network is made of fibers selected from a group consisting of polyamide (or polyaramide) fibers such as those sold under trademark KEVLAR® (DuPont, Richmond, Va.), polyethylene fibers, and liquid crystal polymer fibers such as those sold under the trademark VECTRA® (Celanese, Germany). Preferably, the basket portion is positioned distal to the matter that needs to be removed and, then, is gently withdrawn to retrieve the matter. In one embodiment, the struts aid in the retrieval of the basket portion by allowing it to be collapsed and forced down into a retrieval catheter (not shown).

In one embodiment shown in FIG. 3G, the polymer in its expanded configuration may comprise a formation of a plurality of outwardly extending spears 39. The spears may have a spiral configuration as demonstrated in FIGS. 3G (ii) and (iii).

The polymer 20 may be capable of storing torque energy when in compressed configuration and releasing it in the expanded configuration, much the same way that a twisted rubber band provides a transient surge of energy for a model airplane (i.e., the potential energy stored in the wound-up rubber band powers the plane's propeller). Accordingly, in one embodiment shown in FIG. 3H, a band of expandable polymer 20 (FIG. 3H (i)) is wound-up (FIG. 3H (ii)) and unwinding of the band provides torque to drive a microdevice 37, optionally attached at the distal end 14 of the carrier, for a predetermined time (FIGS. 3H (iii)-3H (iv)). The microdevice may be a tiny propeller, a screw, an auger, or other small device. In one embodiment, the microdevice is capable of dissolving or fragmenting a clot or atheromatous plaque or debris. In another embodiment, the microdevice assists in retrieval of the matter.

Optionally, the polymer 20 may be a temperature sensitive foam or a polymer fiber band. The band may be wound up at a temperature above Tg and then cool down below Tg to stabilize the polymer in the twisted configuration (FIG. 3H (iii)) and to store its potential energy in a stable form. When the twisted band is placed into the environment with temperature above Tg, the polymer is activated and releases the torque stored in the twisted band. In one embodiment, the device has an external source of heat, such as the resistive heater 22 described above, for activating the foam. Optionally, the polymer may be a foam reinforced by fibrous resilient material.

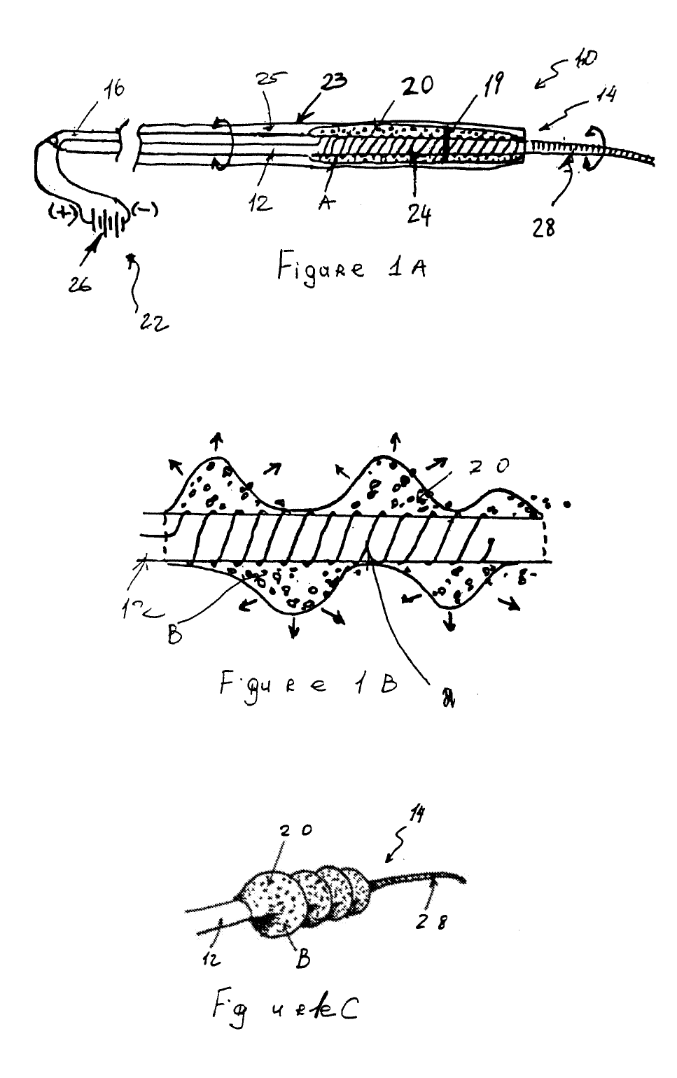

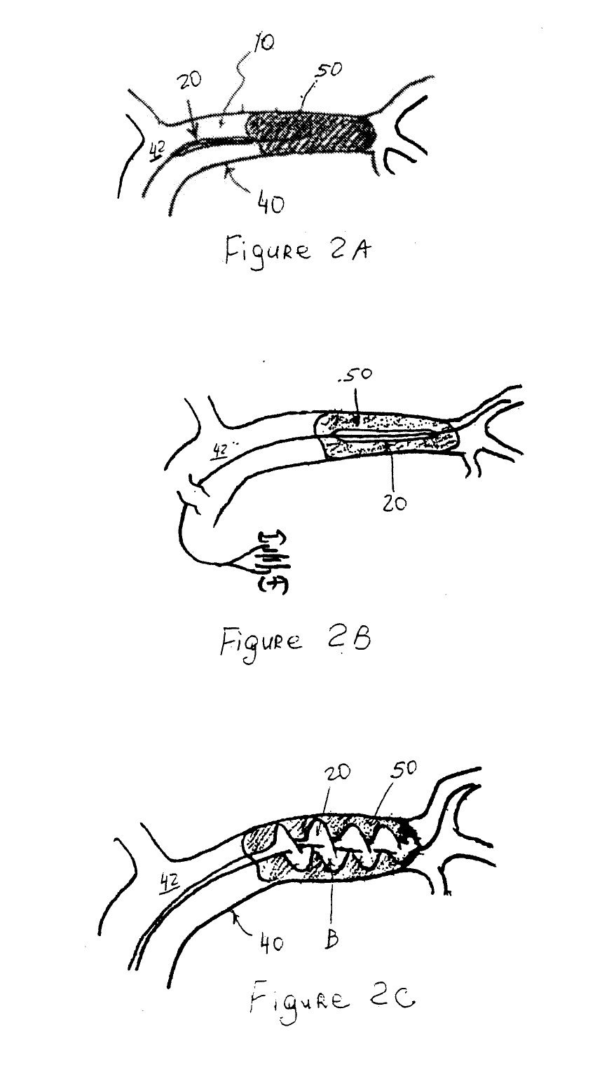

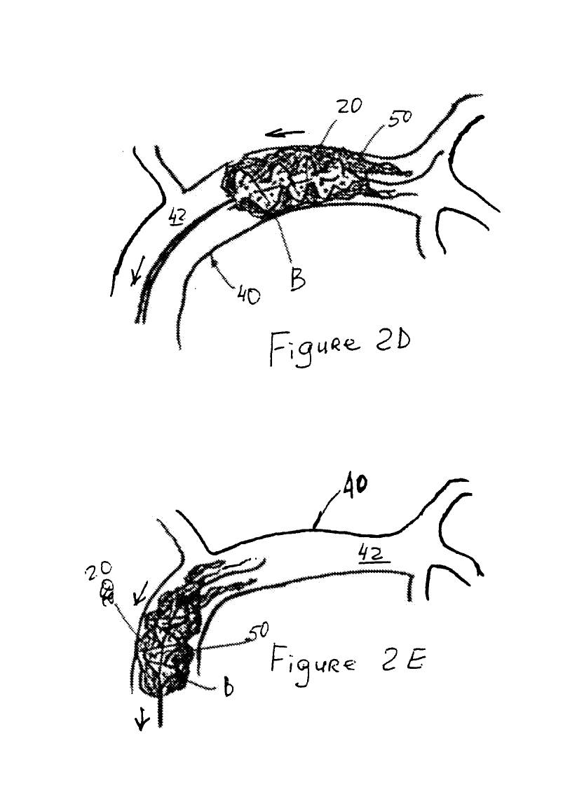

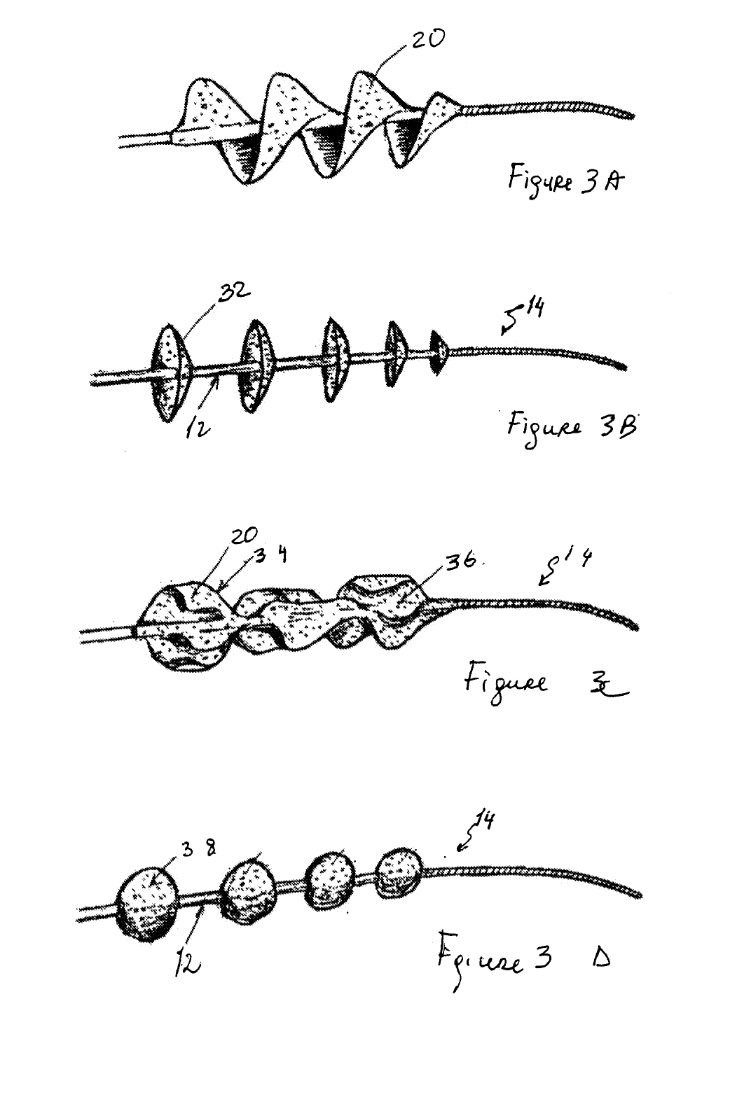

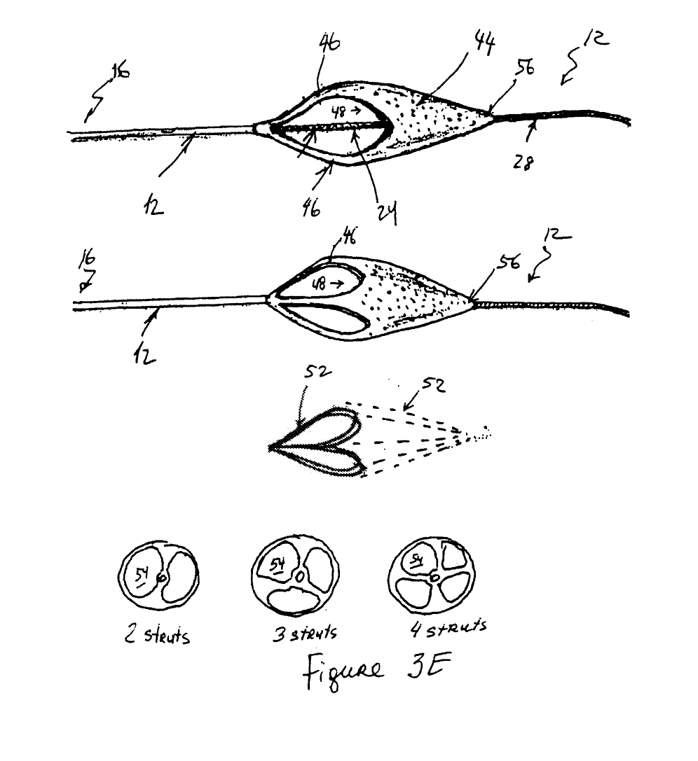

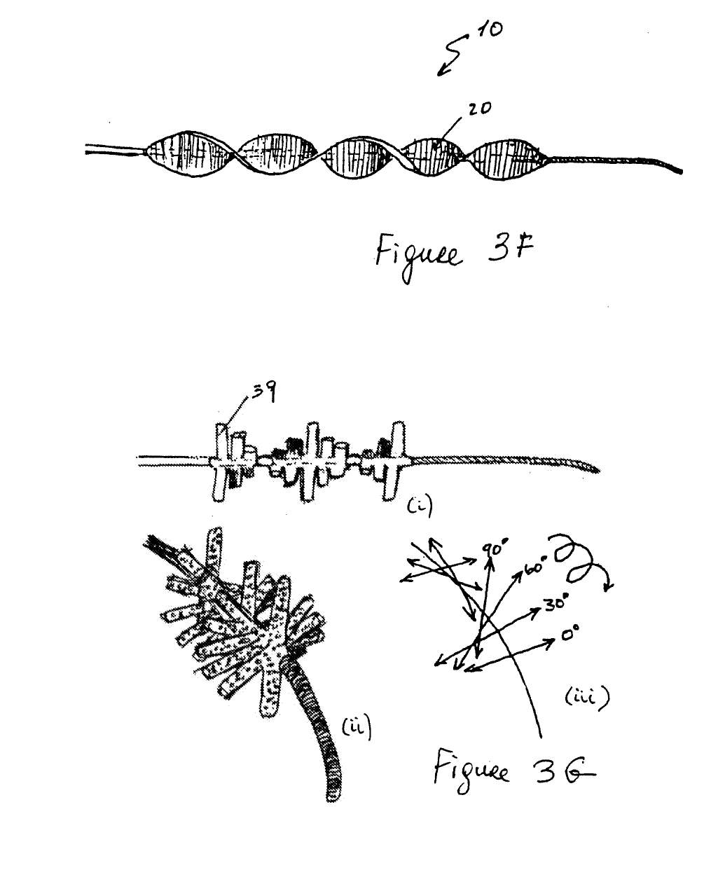

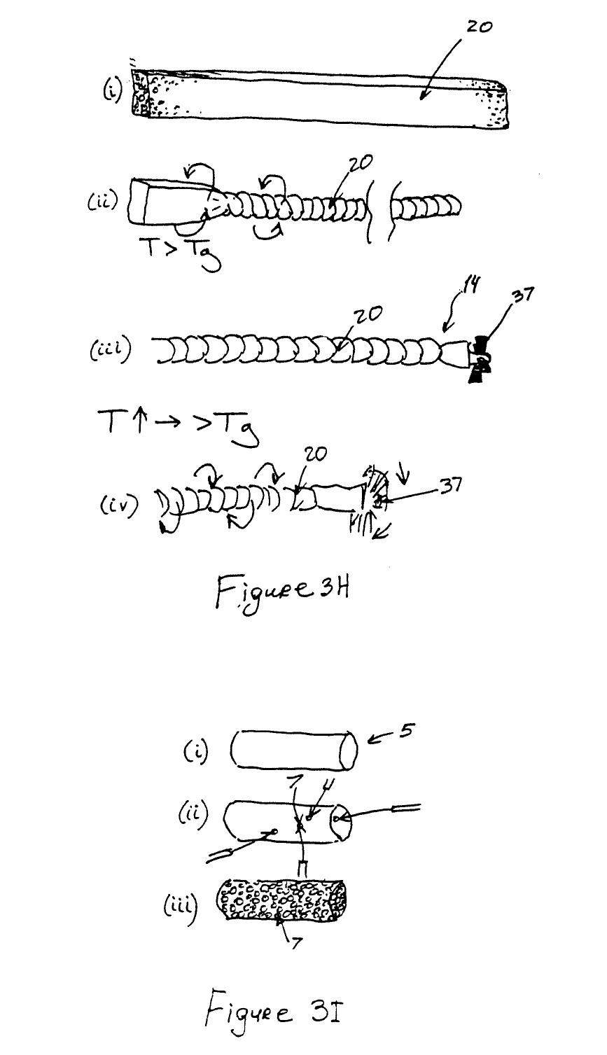

Referring to FIG. 4A, the device of the present invention may also include a thin polymer coating 60 applied to the radially expandable polymer. The coating may be used to prevent fragmentation of the expandable polymer. The coating may also be used to impart desirable physical and chemical properties. For example, in one embodiment, the coating has hydrophilic and/or lubricious properties to aid in advancement of the device inside or through the body cavity. In another embodiment, the coating is used to provide a magnetic field, a positive charge, a negative charge, or their combination to the expandable polymer. In one embodiment, other portions of the device are coated to provide a desirable physical or chemical property.

For example, a magnetically or electrically charged surface of the device may advantageously allow to attract or repel matter inside the body cavity. Alternatively, the expandable polymer itself may provide a desirable surface charge, magnetic field, or other desirable physical or chemical properties. The charge or magnetic field may be an intrinsic property of the polymer, produced by chemical modification of the polymer's surface, or induced by application of an external energy or a source of magnetism. In one embodiment, the charge is induced by an external electrical source or a thermocouple located inside the device. In another embodiment, a magnetic field is created by a fixed permanent magnet or an electromagnet located in the distal portion 14 of the device 10. The electromagnet may be induced by an electric current applied through wires running through the device as seen in FIG. 1A. Depending on the amount of current applied, the configuration of the coils, and the resistive nature of the wire, any combination of resistive heat generation and magnetic field generation may be accomplished.

In one embodiment, the coating is made of an semipermeable elastomeric material such as latex, PVC, silicone rubber, and silicone modified styrenic thermoplastic elastomers sold under the trademark C-FLEX® (Consolidated Polymer Technologies, Inc., Clearwater, Fla.). The coating may be in a form of a sleeve running the length of the device. When a hydrogel is used, the sleeve may advantageously provide a means of injecting a triggering fluid for initiating expansion of the hydrogel. Optionally, the expandable polymer or the optional coating may contain a medical composition that prevents thrombus formation on the expandable polymer. In one embodiment the medical composition comprises heparin and or an anti-platelet adhesion agent to help prevent thrombus formation.

Referring to FIG. 1A, the device may further include radiopaque markers 19 (such as platinum) or a material (such as barium sulfate) that will allow the operator to determine fluoroscopically the location of the device. Also, radiopaque markers may be incorporated into the expandable polymer to allow the operator to see whether the expandable polymer is compressed or expanded configuration.

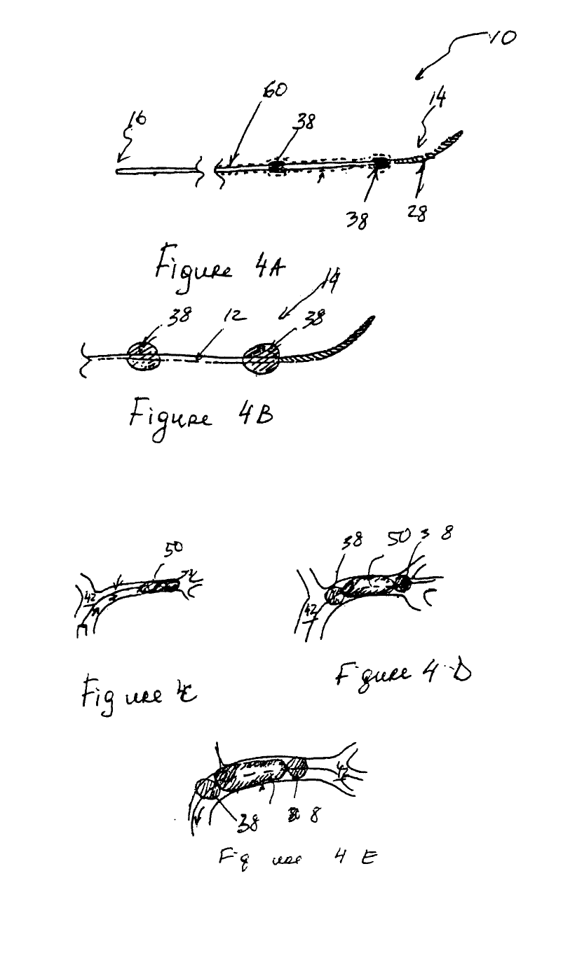

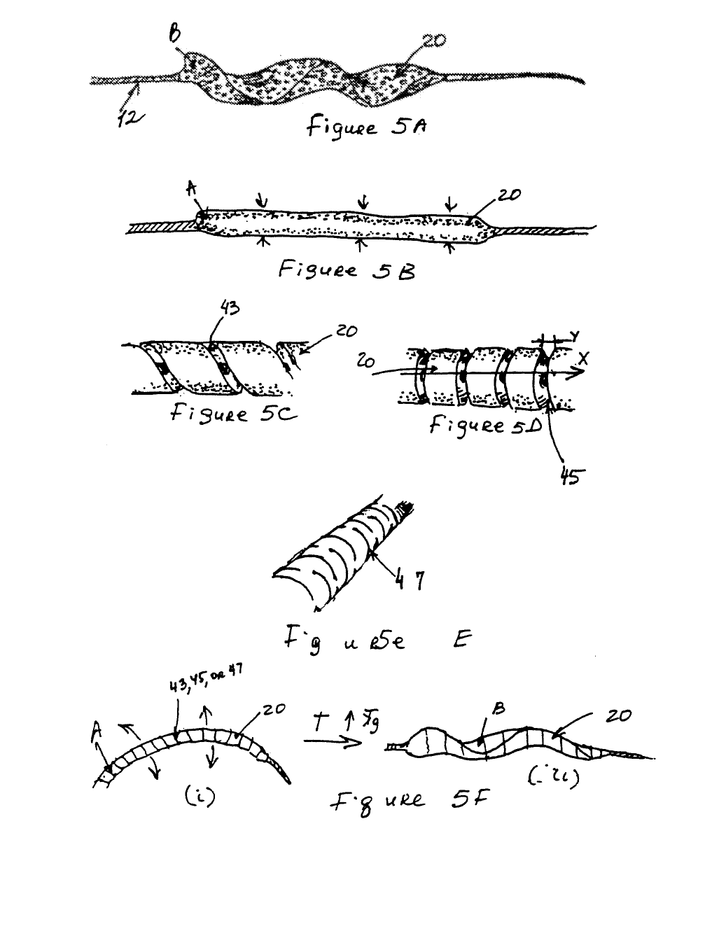

When in the compressed configuration, the expandable polymer may have a reduced flexibility, which may negatively affect maneuverability of the device. Referring to FIGS. 5A-5F, the compressed polymer may be etched or machined to create at least one feature imparting a desired level of flexibility to the carrier with the polymer in the compressed configuration. For example, the feature may be a cut, grove, slot, or indent. In one embodiment, a desirable shape A of expandable polymer 20 is created and attached to the carrier 12. Then, the expandable polymer 20 is heated above Tg and compressed to form a compressed configuration A (FIG. 5B). The expandable polymer 20 retains its compressed figuration until it is exposed to a temperature above Tg.

To improve flexibility and maneuverability of the expandable polymer, cuts, groves, slots, or other features are created using a laser beam, a mechanical blade or other suitable tool. In one embodiment, a continuous spiral cut 43 is formed along the length of the expandable polymer (FIG. 5C). In another embodiment, a plurality of cuts or slots 45 are formed perpendicularly to a long axis X of the carrier, with each slot or cut being offset circumferentially by a distance Y from an immediately preceding slot or cut. As shown in FIG. 5E, in another embodiment, repeating orthogonal cuts 47 may be made to create a complex multiple cut pattern. These and other features afford a greater flexibility to the compressed polymer (FIG. 5F (i)). Yet, when expanded, the expandable polymer substantially returns to its pre-cut expanded configuration B (FIG. 5F(ii)).

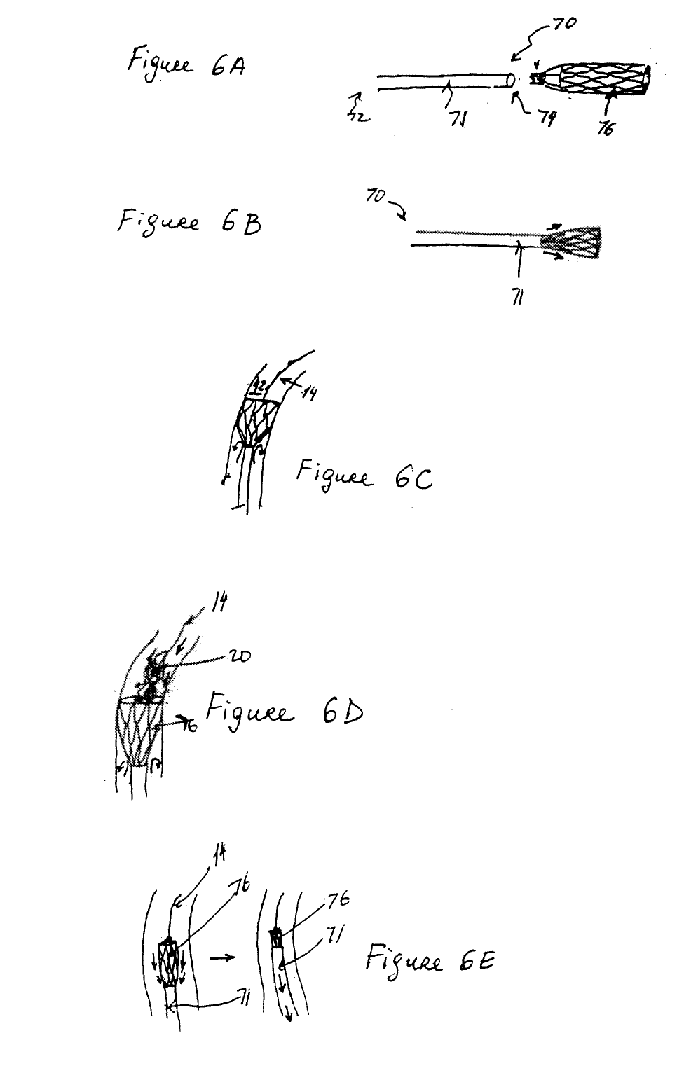

When the obstructing matter is captured by the device of the present invention, it is highly desirable to remove it from the body in a manner that would minimize the risk of its fragmentation or loss. In one embodiment illustrated in FIGS. 6A-6E, this risk is mitigated by utilizing a retrieval element 70 adapted for positioning inside a body cavity, such as a lumen 42 of a blood vessel. The retrieval element may comprise a guiding catheter 71 with a proximal end 72 and a distal end 74. The distal end 74 comprises a self-deploying expandable sleeve 76. The retrieval element has a channel that extends through an entire length of the guiding catheter 71 and the expandable sleeve 76. The distal portion 14 of the carrier 12 is slidably positioned within and adapted to move through the channel into the body cavity. Preferably, the expandable polymer 20 in its expanded configuration is capable of being at least partially retrieved into the expandable sleeve 76 as shown in FIG. 6D.

Optionally, as shown in FIG. 6E, the sleeve is capable of packaging the entire radially expandable polymer in its expanded configuration inside the sleeve. In one embodiment shown in FIGS. 6C-6E, sleeve 76 in its expanded form, advantageously, blocks antegrade blood flow and creates retrograde blood flow toward the open sleeve.

Any construction of the sleeve 76 is acceptable, as long as it is self-deploying and expandable. In one embodiment shown in FIGS. 6A-6E, the sleeve comprises a wire core in a form of a plurality of wire ring components forming a net-like configuration. Such multiple wire ring components may be welded together at several points to provide some flexibility of the design. Very thin (0.004″-0.008″ diameter) wire may be used. The wire may be made of a metal such as titanium or an alloy, such as nitinol, ELGILOY®, Ni/Co/Cr/Mo/Fe alloy (Elgiloy Limited Partnership), and steel. Optionally, a thin cylindrical polyurethane or PTFE sleeve may be attached to the wire core by adhesive application, small sutures, “sandwiching” the wire rings between two thin polymer layers, or some other suitable method.

Preferably, as shown in FIG. 6E, the expandable sleeve may be contained in its collapsed configuration within the distal end 74 of the guiding catheter 71 (e.g., 8-9F guiding catheter) used for the introduction of the device 10 of the present invention into the lumen of the blood vessel. Once the device 10 is withdrawn into the open sleeve 76 with its captured material (FIG. 6D), the sleeve is withdrawn back into the guiding catheter 71 (FIG. 6E), thus, securely packaging the device 10 and the captured matter to allow their safe retrieval from the body.

Referring to FIGS. 6F-6G, alternative designs are possible for the expandable sleeve. For example as shown in FIG. 6F, the sleeve may comprise a plurality of right-angle loops 81 attached to a pusher/retraction wire 82 and having an attached cone-like polymer sleeve 84. In another embodiment shown in FIG. 6G, multiple rings 86 made of a shape-memory material and having progressively enlarging diameters are joined at opposite ends a and b. A cone-like polymer sleeve 84 is attached to the rings. Both of these designs may be contained in a collapsed state within the distal length of the guiding catheter and would be deployed by pushing them out of the end of this catheter. After the device 10 with the captured matter is pulled back into the sleeve 76, the sleeve is collapsed by pulling it back into the catheter, thus allowing safe retrieval of the captured material.

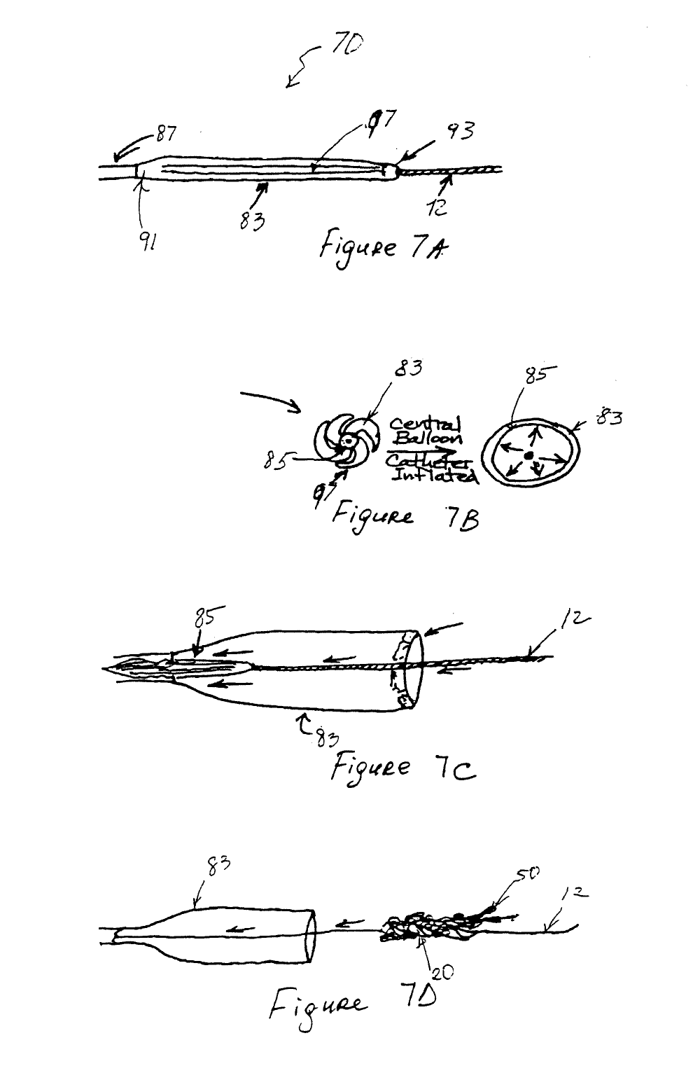

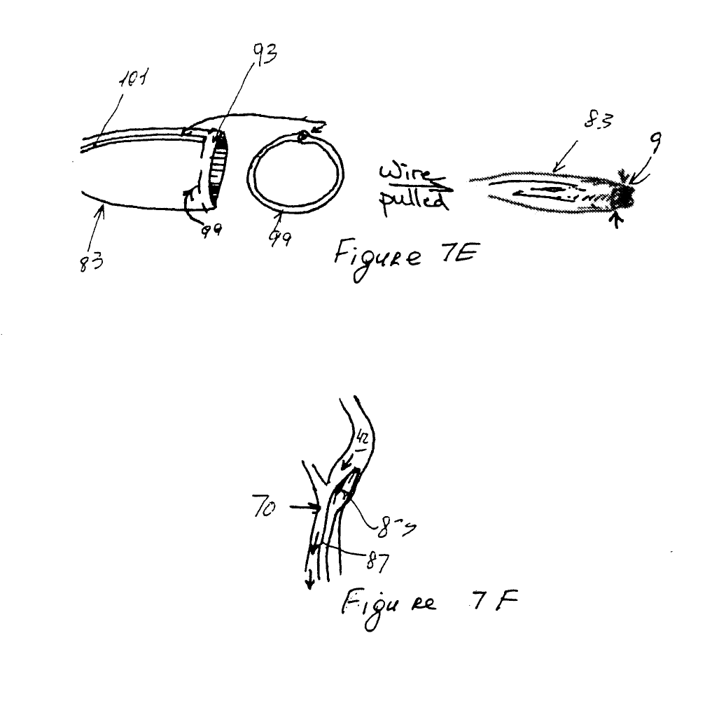

Referring to FIGS. 7A-7F, retrieval element 70 may have an inflatable removable balloon-like structure (referred to as balloon) 85 for expanding the sleeve 83. In this embodiment, the expandable sleeve has a proximal end 91 and a distal end 93. The proximal end may be attached to a non-expandable shaft 87. In one embodiment, the shaft is a 7-8F shaft. The distal end 93 may be tapered. Preferably, at least the distal end of the expandable sleeve is made of an elastomeric material such as SILASTIC® (Dow Corning, Midland, Mich.) or C-FLEX® (Consolidated Polymer Technologies, Inc., Clearwater, Fla.) material. The balloon 85 is positioned concentrically inside the expandable sleeve 83. In one embodiment, the elongated carrier 12, such as 0.035-0.038″ guidewire, is slidably positioned through the center of the balloon 85. As shown in FIG. 7B, the expandable sleeve 83 may be folded to form folds or “wings” 97 and wrapped tightly like an angioplasty balloon. When the balloon 85 is inflated, it expands the sleeve 83 from within. Then, as shown in FIG. 7C, the balloon 85 may be deflated and removed through the non-expandable shaft 87. Referring to FIG. 7D, the expanded sleeve 83 accommodates, at least partially, polymer 20 in its expanded configuration with the captured matter 50. Referring to FIG. 7F, the retrieval element 70 with the trapped matter may then be removed from the body cavity 42.

Referring to FIG. 7E, the distal end 93 of the sleeve 83 may be optionally contracted after the expandable polymer with the captured matter is retrieved into the sleeve. In one embodiment, a loop structure 99 is placed circumferentially at a distal end 93 of the expandable sleeve 83. Another longitudinal structure 101 is placed longitudinally through a separate lumen in the sleeve 83 and is connected to the loop structure 99. The loop structure and the longitudinal structure may be made of any flexible material such as a metal wire, purse-string, or radiopaque suture. When the longitudinal structure 101 is retracted, the distal end 93 of the sleeve 83 contracts and captures the trapped matter 50.

Referring to FIGS. 1-4, in still another aspect, the present invention provides another device for retrieving a matter 50 from a body cavity such as a lumen 42 of a blood vessel. The device comprises an elongated carrier 12 having a distal portion 14 adapted to move through the lumen and a radially expandable polymer 20 circumferentially attached to the distal portion 14 of the carrier. The polymer is adapted to move through the matter while having a compressed configuration and capable to transition to an expanded configuration to engage the matter for retrieval from the lumen. In this embodiment of the invention, the transition of the polymer is triggered by a physiological stimulus.

The present invention further provides a method of retrieving a matter from a lumen of a blood vessel. The method comprises (a) providing a device with a retrieval element having a balloon-expandable sleeve described above; (b) positioning the retrieval element inside the body cavity; (c) advancing the distal portion of the carrier through the channel of the retrieval element and the balloon-expandable sleeve of the retrieval element into the body cavity; (d) moving the distal portion of the carrier through or around the matter; (e) allowing sufficient time for the expandable polymer to transition from the initial compressed configuration to an expanded configuration whereby trapping the matter; (f) inflating the balloon whereby expanding the sleeve; and (g) retrieving, at least partially, the carrier with the expandable polymer in its expanded configuration into the expanded sleeve.

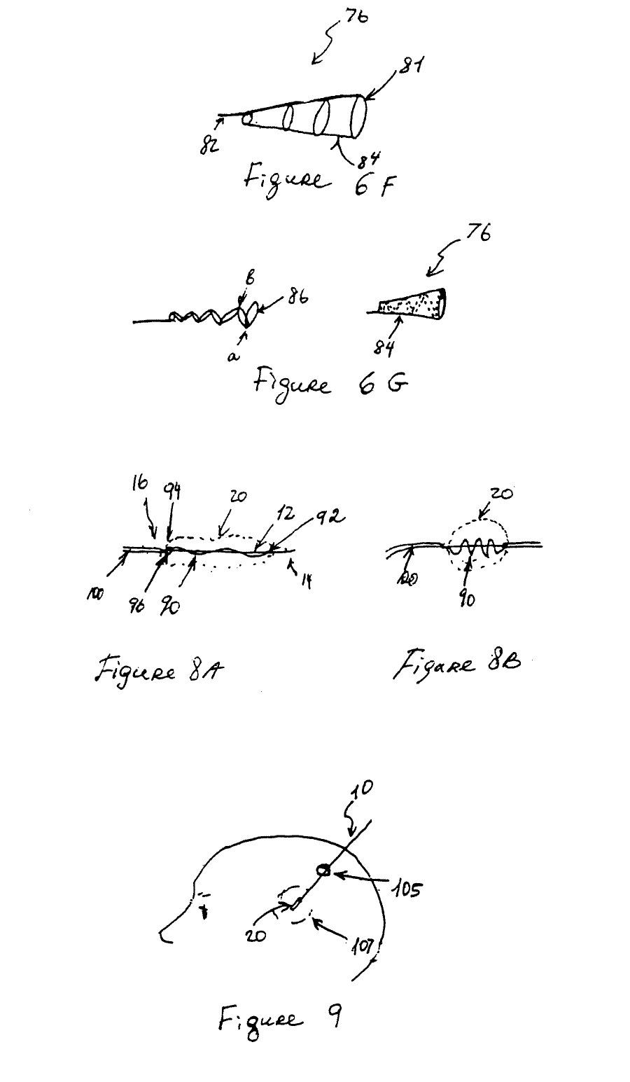

As shown in FIGS. 8A and 8B, the profile of the expandable polymer used to capture the obstructing matter could be significantly enlarged by a wire coil 90 running through the expandable polymer 20 along the carrier 12. The wire coil 90 has a first end 92 fixedly attached by welding or some other means to the distal portion 14 of the carrier and a second end 94 movably attached to the proximate portion 16 of the carrier 12. The proximal end 94 of the coil may have a small loop 96 or a coaxial metal or plastic tube (not shown) keeping the coil attached to the carrier 12, but allowing it to slide freely along the carrier 12.

The device further comprises a barrier circumferentially attached to the carrier, wherein the barrier prevents movement of the second end when the carrier is pulled back, whereby the coil radially expands. For example, the device may have a more proximal microcatheter 100 or hypotube such that when the central wire is pulled back through this microcatheter or hypotube, the free-sliding proximal connection of the coil would be held stationary, thus foreshortening the coil and increasing its diameter (FIG. 7B).

Referring to FIGS. 2A-2E, the present invention further provides a method of retrieving a matter 50 from a body cavity such as a lumen 42 of the blood vessel. The method comprises (a) providing the device 10 described above; (b) positioning the device inside the lumen 42 of the blood vessel; (c) allowing a sufficient time for the expandable polymer 20 to expand and engage the matter 50 from within; and (d) removing the device whereby removing the matter.

Referring to FIGS. 4A-4D, the present invention further provides a method of retrieving a matter 50 from a body cavity such as a lumen 42 of a blood vessel. The method comprises (a) providing the device 10 described above and shown in FIG. 4A, the device having at least two isolated formations 38 of radially expandable polymer attached to the distal portion 14 of the carrier 12; (b) positioning the device inside the lumen 42 of the blood vessel; (c) passing at least one formation 38 through or around the matter 50; (d) allowing a sufficient time for the e isolated formations 38 to expand whereby trapping the matter between the formations; and (e) removing the device whereby removing the matter.

Referring to FIGS. 2A-2E and 4A-4D, the present invention further provides a method of retrieving a matter 50 from a body cavity such as a lumen 42 of a blood vessel. The method comprises (a) providing the device 10 described above; (b) positioning the device inside the lumen 42 of the blood vessel; (c) allowing a sufficient time for a physiological stimulus to act on the expandable polymer to cause its transition from the initial compressed configuration to an expanded configuration whereby engaging the matter in a way that allows its removal; and (d) retrieving the device thereby removing the matter.

As discussed in more detail above, the physiological stimulus may be body temperature, blood pH, an ion concentration in blood, and blood composition. The polymer may be a hydrogel or a foam. In one embodiment, the polymer is a hydrogel and the transition of the hydrogel into the expanded configuration is triggered by a hydration of the hydrogel inside the blood vessel.

In yet another aspect, the present invention provides a method of localized delivery of a therapeutic agent. The method comprises (a) providing a removable device having a carrier with a distal portion and a radially expandable polymer circumferentially attached to the distal portion of the carrier, wherein the expandable polymer has an initial compressed configuration; (b) advancing the distal portion of the carrier to a site in a body; and (c) allowing sufficient time for the expandable polymer to transition from the initial compressed configuration to an expanded configuration delivering the therapeutic agent.

There is no limitation on a type of the site in the body to which this method could be applied. The site may be a lumen, such a lumen of a blood vessel, a lumen of the alimentary tract including esophagus, stomach, small and large bowel, anus and rectum, or a lumen of the genitourinary system including renal pelvis, ureter, urethra, spermatic cord, fallopian tubes, a cavity, such as the ventricles and cisterns of the brain as well as the urinary bladder, cysts, vagina, uterine cavity, pseudocysts, abcesses, fistulae, surgically created conduits and cavities or a solid tissue, such as liver, spleen, pancreas brain, bone, muscle, tumors, testis, ovaries, uterus, lymph nodes.

For example, as shown in FIG. 9, the device 10 of the present invention may be placed in proximity of a brain tumor 107 through a burr hole 107 drilled in the skull of a patient. The device may be placed with the assistance of CT, MR, stereotactic, or other means. The expandable polymer 20 is then activated to release a suitable therapeutic agent. The expandable polymer may be activated by a physiological and/or externally applied stimulus as discussed in detail above.

The foregoing is meant to illustrate, but not to limit, the scope of the invention. Indeed, those of ordinary skill in the art can readily envision and produce further embodiments, based on the teachings herein, without undue experimentation.

### EXAMPLE 1

Under fluoroscopic and/or digital roadmap imaging, an appropriate guiding catheter (with or without a distal occlusive balloon) is navigated into the cervical artery (i.e., the internal carotid or vertebral artery), serving the distal intracranial circulation affected by the occlusive thromboembolus or intraluminal foreign body (“target”). Coaxially through this guiding catheter, an appropriate microcatheter (with O.D.=2-3 F and I.D.=0.018″-0.025″) is maneuvered over a steerable microguidewire (approximate diameter=0.014″) under fluoroscopic guidance into the affected intracranial artery just proximal to the target. The aforementioned microguidewire is removed from the microcatheter, and a device with foam circumferentially attached to a wire, in accordance with one embodiment of the present invention, is advanced coaxially through the microcatheter. Using digital roadmap imaging and/or regular fluoroscopy, the device is then navigated through the target.

Next, the heater controller attached to the proximal external end of the device is activated. The resistive heater subjacent to the compressed foam layer raises the temperature of the compressed foam segment to the Tg for several minutes. The foam begins to expand, assuming its expanded configuration as revealed by radiopaque markers or material within the foam segment. The foam expands into the target and engages the target from within.

The target is then dragged from the occluded vessel thus restoring blood flow to the distal distribution of this vessel. Under fluoroscopic guidance, the retrieval device with its captured target is carefully withdrawn into the aforementioned cervical artery and into the guiding catheter. If an inflated occlusive balloon tip is used, it ensures retrograde blood flow within the cervical vessel (i.e., toward the guiding catheter tip), thus facilitating successful removal of the retrieval device and the trapped target.

### EXAMPLE 2

A device in accordance with one embodiment of the present invention shown in FIGS. 4A-4D is used. The device has two isolated formations of a hydrogel attached to a wire. Under fluoroscopic and/or digital roadmap imaging, the device is advanced into a lumen containing a matter to be removed. The device is maneuvered around the matter so that the matter is located between two formations. The device is left in place for several minutes. The hydrogel begins to swell with the absorption of ambient water so as to transition from a compressed into an expanded configuration. The matter becomes trapped between two expanded formations of the hydrogel and dragged from the occluded vessel thus restoring blood flow to the distal distribution of this vessel.

### EXAMPLE 3

In this example, an apparatus in accordance with the present invention is shown in FIGS. 10-12 as having a central matter retrieval device (FIG. 10), to be fitted in a delivery catheter device (FIG. 11), and a device retriever (FIG. 12) to be used together with the central matter retrieval device and the delivery catheter device.

Referring first to FIG. 10, the central matter retrieval device has an elongated body that runs from a proximal end 101 to a distal end 102. The elongated body may be constructed of a wire material commonly used in the art. It may also have variable thickness. One or more expandable reticular foam discs 103 are disposed on the body on the distal end, for example, between the locations marked 106 and 108. A polymer sleeve 105 may be optionally included as a spacer between the discs to prevent their sliding. The thickness of the proximal end 101 is preferably from about 0.010 to about 0.035 inch, more preferably from about 0.014 inch to about 0.027 inch. A skilled person in the art will readily recognize that the choice of the thickness will depend on the application, the desired flexibility of the retrieval device and other factors. Along the length of the device body, variable thickness may be chosen to define different segments of the body. For example, the thickness at position 106 may be from 0.010 to 0.035 inches. In one embodiment, the thockness is 0.014 inch. The thickness of distal end 102 may be from 0.003 to 0.018 inches. In one embodiment, it is 0.010 inches. The reticular foam discs, when expanded, should have a diameter compatible with the size of bodily cavity such as a blood vessel. In one embodiment, their diameter is from about 1 to about 14 mm, more preferably from about 2 to about 3 mm.

Referring now to FIG. 11, the delivery catheter device has a hollow elongated body with a proximal end 111, a parking segment 113, a proximal wire side-port 114, and a distal end comprising a placement wire 112. The placement wire enters from the side-port 114, slidably fits through the lumen of the hollow device body and exits at the distal end of the device body forming a guide-wire overhang. The placement wire 112 may have a thickness of about 0.010 to about 0.035 inch. In one embodiment, it is about 0.014 inch thick.

The distal portion that guides the placement wire, also called monorail or rapid-exchange distal segment (the portion of the device starting from the position marked by 114 to the distal end), may be between about 2 to about 30 cm, preferably, between about 20-24 cm in length.

The middle segment shown in FIG. 11 (park segment) houses the central matter retrieval device shown in FIG. 10. The compressed central matter retrieval device is parked in the parking segment before use and during insertion of the catheter into a patient.

On the proximal end, there are scoring (e.g. notch cut onto the surface) 115 on the device body to allow it to be peeled away. In general, the device body is a catheter that houses the placement guide-wire 112 and the central matter retrieval device of FIG. 10. This allows compact packing of delivering the device to the desired location. The scoring are designed such that it enables easy peeling off of the delivery device body (e.g. a catheter) once the body is in place so that the proximal end of the central matter retrieval device of FIG. 10 may be accessed.

FIG. 12 shows an exemplary retriever device of the present invention having an expandable trumpet-like structure at the distal end for retrieving and securing a central matter retrieval device. Referring to FIG. 12A, the device retriever has an elongated body with a proximal pusher portion 121, an introducer sleeve 123, a trumpet-like structure 126, and a guide-wire 122. When in operation, the guide-wire 122 is typically the device body 101 of the central matter retrieval device of FIG. 10, although this is not a requirement. The trumpet-like structure 128 functions as a catcher for securing the central matter retrieval device of FIG. 10. The trumpet-like structure 126 may be constructed from a variety of suitable materials, including, but not limited to soft polyurethane layer mounted on Nitinol wire skeleton.

The introducer sleeve is slidably disposed on the proximal end of the device body and may be slid along the body in the directions indicated by 124.

FIG. 12B shows a method for assembling the device retriever of FIG. 12A prior to use according to one embodiment of the present invention. On the left hand side of FIG. 12B, the trumpet-like structure in its expanded configuration is fitted into one end of the introducer sleeve 123. By advancing the introducer sleeve 123 in the direction indicated while keeping the trumpet-like structure stationary, the trumpet-like structure is collapsed and loaded into the introducer sleeve 123. The right hand side of FIG. 12B shows the trumpet-like structure in a collapsed configuration after being loaded into the introducer 123. In operation, the pre-loaded device retriever is slidably mounted on the body of the central matter retriever 101 and advanced towards the distal end for catching the reticular foam discs and the retrieved matter.

FIG. 13 illustrates the function of the reticular foam discs in a device of the present invention. In FIG. 13a, the reticular foam discs are initially in a collapsed configuration and maneuvered into position inside a bodily cavity such as a blood vessel with the assistance of a 3F delivery catheter. In the figure, the catheter has encountered a blood clot. The 0.014 inch placement wire is used to bore through the blood clot to prepare the pathway for insertion of the collapsed reticular foam discs.

In FIG. 13b, the 0.014 inch placement wire is withdrawn from the catheter to clear the pathway. The reticular foam discs collapsed and retained in the delivery device (i.e. the catheter) are advanced forward from the parking segment into the blood clot.

In FIG. 13c, the scored proximal end (proximal end outside of the patient) is peeled away while holding the 0.014 inch wire stationary, thereby, unsheathing the foam discs to allow them to expand within the blood clot to engage the clot.

Next, in FIG. 13d, the core wire is rotated repeatedly to rotate and entangle the blood clot with the reticular foam discs. While the figure illustrates blood clot as an example, it is to be understood that any other foreign objects (e.g. an aberrant or lost endovascular coil) may be entrapped and retrieved by the foam-disc.

In FIG. 13e, the entangled blood clot is gently withdrawn and is pulled into the cervical internal carotid artery (or the vertebral artery when clot retrieval is performed is performed within the basilar artery).

At this point, the trumpet-like receiver is mounted and advanced in a compressed state through the guiding catheter over the 0.014 inch wire.

Next, in FIG. 13f, the catheter is withdrawn while holding the receiver stationary. After the catheter is withdrawn, the initially collapsed trumpet-like receiver springs open to assume a trumpet-cone configuration. The trumpet cone has an opening facing the cephalad, which acts to block antegrade ICA blood flow and at the same time creates a retrograde blood flow above the trumpet.

Finally, in FIG. 3g, the foam clot retriever with the entrapped thrombus is withdrawn into the trumpeted end of device retriever-retrograde ICA flow helps ensure that all clot fragments are captured in trumpet. The device retriever is then withdrawn into guiding catheter, thus, collapsing trumpet and safely encapsulating the trapped thrombus.

In this embodiment, the device retriever is vital since it ensures the removal of all captured clot and does not allow clot fragmentation that can result in distal re-embolization into previously unaffected cerebral arteries (as can occur during use of the Concentric Merci device). The 0.014″ proximal wire of the foam clot retriever can also act as a guide wire for other devices, e.g., angioplasty balloon catheters particularly if a proximal stenosis is preventing retrieval of captured clot. As noted above, this system may be used for foreign body (especially coils) retrieval and within a variety of bodily cavities, including, but not limited to, carotid and vertebrobasilar arterial systems, iliac artery, superficial femoral artery and its branches, renal artery, visceral arteries, pulmonary artery and its branches, coronary arteries, bypass vessels, and intra- and extracranial venous systems.

The reticulated foam collections responsible for entrapping the clot can be made in a variety of shapes and lengths besides the 2-disc configuration presented above. FIG. 14 illustrates several exemplary shapes that may be beneficially employed. Other shapes and designs are also possible depending on the desired effect and the target to be engaged and retrieved.

In some other embodiments, the present invention may also be adapted to “unclog” a long vascular channel such as the femoral/illiac veins during deep venous thrombosis (DVT). FIG. 15 illustrate just such an application.

In FIG. 15, a long foam cylinder (on wire) device is introduced via sheath in popliteal vein. The leading wire is navigated through thrombosed iliofemoral venous system (cephalad, in the direction of the venous valves). The distal lead wire is captured using an endovascular snare (or similar device) and is pulled through expanded trumpet device extending from a guiding catheter in iliac vein or IVC above region of DVT. Now one has achieved control over both ends of clot retriever. Using rotation and a push-pull technique, the clot retriever is advanced through the thrombosed venous segments. Finally, the trailing wire end is pulled through the popliteal vein introducer sheath, through the de-clotted vessel and into the trumpet-ended device retriever above.

It will be apparent to those skilled in the art that various modifications and variations can be made in system and methods of the present invention without departing from the spirit or scope of the inventions. Thus, it is intended that the present invention cover modifications and variations of this invention that come within the scope of the appended claims and their equivalents.

What is claimed is:

1. A system for removing a matter from a body cavity of a patient, comprising:
a delivery catheter device having a proximal end, a middle park segment, and a distal end, wherein the catheter device is capable of allowing a guidewire to slidably fit within its hallow inner space, and wherein the distal portion is adapted for positioning inside the body cavity;
a central matter retrieval device having an elongated body with a proximal end and a distal end, wherein the distal end has one or more formations of a shape memory polymer disposed thereon and wherein the central retrieval device is slidably positioned within the park segment of the delivery catheter device; and
a device retriever having an elongated body with a pusher proximal portion and an introducer sleeve slidably mounted thereon.

2. The system of claim 1, wherein the shape memory polymer is a foam selected from a group consisting of polyurethane, polyethylene, polyethylene terephthalate, polyisoprene, styrene-butadiene copolymers and other styrenes, copolyester, ethylene-vinylacetate and other ethylene copolymers, polyacrylamide gel, polyacrylic acid and other acrylates, norbornane, polystyrene, and polynorbornene.

3. The system of claim 1, wherein the park segment of the catheter delivery device form a sheath that covers the central matter retrieval device.

4. The system of claim 1, wherein the transition of the shape memory polymer between the compressed configuration and the expanded configuration is triggered by withdrawing the catheter to unsheathe the shape memory polymer.

5. The system of claim 1, wherein the polymer comprises a predetermined amount of a therapeutic agent.

6. The system of claim 5, wherein the therapeutic agent is released when the polymer transitions to the expanded configuration.

7. The system of claim 5, wherein the therapeutic agent is selected from a group consisting of anti-thrombogenic, thrombolytic, anti-proliferative, anti-spasmodic, anticoagulant, anti-platelet adhesion drugs, endothelial cells, and gene vectors.

8. The system of claim 2, wherein said foam is a shape memory foam having an original expanded configuration that can be compressed at a temperature above a glass transition temperature, Tg, to form the compressed configuration, wherein the foam retains its compressed configuration at a temperature below the Tg but returns substantially to its original expanded configuration when it is exposed to a temperature above the Tg.

9. The system of claim 8, wherein the Tg is above a body temperature and the device further comprises an external source of heat.

10. The system of claim 9, wherein the external source of heat is selected from a group consisting of electrical resistance, inductive, optical, and convective heating elements.

11. The system of claim 10, wherein the electrical resistance element comprises a metal coil or a semi-conductive plastic that is circumferentially attached to the distal portion of the carrier and electrically connected to a controller through an insulated pathway, the controller capable of adjusting a voltage applied to the resistance element to maintain a predetermined temperature.

12. The system of claim 8, wherein the Tg is below a body temperature and the foam spontaneously transitions into the expanded configuration after being exposed to the body temperature for a predetermined time.

13. The system of claim 1, wherein the distal portion of the elongated carrier further comprises a steerable tip.

14. The system of claim 13, wherein the steerable tip is a shapeable platinum or stainless steel wire.

15. The system of claim 2, wherein the foam has a shape selected from one of those shown in FIG. 14.

16. The system of claim 1, wherein the shape memory foam is configured as one or more reticular foam discs.

17. The system of claim 1, wherein the device retriever comprises a self-expanding trumpet-like structure on the distal end, wherein the trumpet-like structure is capable of being collapsed inside a catheter by sliding the introducer over the trumpet-like structure.

18. The system of claim 17, wherein the trumpet-like structure is constructed from a soft polyurethane layer mounted on a Nitonol wire skeleton.

19. The system of claim 1, wherein the body cavity is selected from a group consisting of lumens of blood vessels, lumens of the alimentary tract, lumens of the genitourinary system, ventricles and cisterns of the brain, urinary bladder, cysts, vagina, uterine cavity, pseudocysts, abcesses, fistuae.

20. The system of claim 1, wherein the body cavity is a blood vessel lumen, the polymer coating carries a positive charge, a negative charge, or both and wherein the coating is capable of attracting or repealing blood clots.

21. The system of claim 1, wherein the matter is a clot, an embolus, calculus, atherosclerotic plaque, loose tissue or neoplasm, inspissated fluid or secretion, or a foreign body.

22. A method of retrieving a matter from a body cavity of a patient comprising:
(a) providing an system according to claim 1;
(b) positioning the central retrieval device inside the body cavity of the patient;
(c) engaging a matter within the body cavity with the central retrieval device;
(d) securing the central retrieval device and the engaged matter using the device retriever; and
(e) withdrawing the system from the patient, thereby removing the matter from the patient.

23. The method of claim 22, wherein the matter is blood clot from deep venous thrombosis.

24. A method for securely and safely removing a clot from a blood vessel, comprising:
(a) providing a system according to claim 17;
(b) positioning the delivery catheter device inside a blood vessel such that the distal end of the catheter passes through the clot and the park segment is positioned inside of the clot;
(c) withdrawing the catheter delivery device to expose the central matter retrieval device and allow the memory shape foam to engage the clot;
(d) deploying the device retriever to secure the central matter retrieval device and the clot, wherein the deploying step involves expanding the trumpet-like structure to capture the central matter retrieval device; and
(e) removing the entire system from the patient, wherein the blood clot is removed together with the system from the patient.
